(19) United States
(12) Patent Application Publication
(10) Pub. No.: US 2023/0292832 A1
(43) Pub. Date: Sep. 21, 2023

(54) AEROSOL PROVISION SYSTEM

(71) Applicant: Nicoventures Trading Limited, LONDON (GB)

(72) Inventors: Steve HUGHES, London (GB); Martyn STANIFORTH, London (GB); David Alan NELSON, London (GB)

(21) Appl. No.: 18/006,637

(22) PCT Filed: Jul. 9, 2021
(86) PCT No.: PCT/GB2021/051763

§ 371 Date: Jan. 24, 2023

(30) Foreign Application Priority Data
Jul. 24, 2020 (GB) 2011517.6

(51) Int. Cl. A24F 40/40; A24F 40/10; A24F 40/50; F16J 15/32

(52) U.S. Cl. CPC A24F 40/40; A24F 40/10; A24F 40/50; F16J 15/32; A24F 40/44

(57) ABSTRACT

There is provided an aerosol provision system including a vaporizer for generating a vapor from an aerosolizable material; an electrode 10 for receiving electrical power; and a sealing member 100, electrically connected to the vaporizer and the electrode, for transferring electrical power between the electrode and the vaporizer, wherein the sealing member is at least partially composed of a heat-resistant and electrically conductive composite material.

### PRIORITY CLAIM

The present application is a National Phase entry of PCT Application No. PCT/GB2021/051763, filed Jul. 09, 2021, which claims priority from GB Application No. 2011517.6, filed Jul. 24, 2020, each of which hereby fully incorporated herein by reference.

### TECHNICAL FIELD

The present disclosure relates to aerosol provision systems such as, but not limited to, substance (e.g. nicotine) delivery systems (e.g. electronic cigarettes and the like).

### BACKGROUND

Electronic aerosol provision systems such as electronic cigarettes (e-cigarettes) generally contain an aerosol precursor material, such as a reservoir of a source liquid containing a formulation, typically but not necessarily including nicotine, or a solid material such a tobacco-based product, from which an aerosol is generated for inhalation by a user, for example through heat vaporization. Thus, an aerosol provision system will typically comprise a vaporizer, e.g. a heating element, arranged to vaporize a portion of precursor material to generate an aerosol in an aerosol generation region of an air channel through the aerosol provision system. As a user inhales on the device and electrical power is supplied to the heating element, air is drawn into the device through one or more inlet holes and along the air channel to the aerosol generation region, where the air mixes with the vaporized precursor material and forms a condensation aerosol. The air drawn through the aerosol generation region continues along the air channel to a mouthpiece opening, carrying some of the aerosol with it, and out through the mouthpiece opening for inhalation by the user.

It is common for aerosol provision systems to comprise a modular assembly, often having two main functional parts, namely a control unit and disposable / replaceable cartridge part. Typically, the cartridge part will comprise the consumable aerosol precursor material and the vaporizer/heating element (atomizer), while the control unit part will comprise longer-life items, such as a power supply, such as a rechargeable battery, device control circuitry, activation sensors and user interface features. The control unit may also be referred to as a reusable part or battery section and the replaceable cartridge may also be referred to as a disposable part or cartomizer.

The control unit and cartridge are mechanically coupled together at an interface for use, for example using a screw thread, bayonet, latched or friction fit fixing. When the aerosol precursor material in a cartridge has been exhausted, or the user wishes to switch to a different cartridge having a different aerosol precursor material, the cartridge may be removed from the control unit and a replacement cartridge may be attached to the device in its place.

Electrical contacts / electrodes are provided on each of the control unit and cartridge for transferring power between the two components. In the case of each electrode on the cartridge, a lead is employed to transfer power from the electrode to the heating element in the cartridge.

A potential drawback in such cartridges is that the lead may become detached from the electrode during use, causing unwanted short-circuits and faulty operation of the cartridge. A potential further drawback for such cartridges, which typically contain liquid aerosol precursor (e-liquid) is the risk of leakage. An e-cigarette cartridge will typically have a mechanism, e.g. a capillary wick, for drawing liquid from a liquid reservoir to a heating element located in an air path / channel connecting from an air inlet to an aerosol outlet for the cartridge. Because there is a fluid transport path from the liquid reservoir into the open air channel through the cartridge, there is a corresponding risk of liquid leaking from the cartridge. Leakage is undesirable both from the perspective of the end user naturally not wanting to get the e-liquid on their hands or other items.

Various approaches are described herein which seek to help address or mitigate some of the issues discussed above.

### SUMMARY

According to a first aspect of certain embodiments, there is provided an aerosol provision system comprising: a vaporizer for generating a vapor from an aerosolizable material; an electrode for receiving electrical power; and a sealing member, electrically connected to the vaporizer and the electrode, for transferring electrical power between the electrode and the vaporizer, wherein the sealing member is at least partially composed of a heat-resistant and electrically conductive composite material.

According to second aspect of certain embodiments, there is provided a cartridge for an aerosol provision system comprising the cartridge and a control unit, wherein the cartridge comprises: a vaporizer for generating a vapor from an aerosolizable material; an electrode for receiving electrical power from the control unit; and a sealing member, electrically connected electrically connected to the vaporizer and the electrode, for transferring electrical power between the electrode and the vaporizer, wherein the sealing member is at least partially composed of a heat-resistant and electrically conductive composite material.

According to a third aspect of certain embodiments, there is provided the use of a sealing member in an aerosol provision system to reduce galvanic corrosion, wherein the sealing member is at least partially composed of a heat-resistant and electrically conductive composite material.

It will be appreciated that features and aspects of the disclosure described above in relation to the various aspects of the disclosure are equally applicable to, and may be combined with, embodiments of the disclosure according to other aspects of the disclosure as appropriate, and not just in the specific combinations described herein.

### BRIEF DESCRIPTION OF THE DRAWINGS

Embodiments of the disclosure will now be described, by way of example only, with reference to the accompanying drawings, in which:

FIG. 1 schematically represents an aerosol provision system comprising a cartridge and a control unit.

FIG. 2A schematically represents a cross sectional view of a cartridge, for use with the control unit from FIG. 1, in accordance with certain embodiments of the disclosure.

FIG. 2B shows a perspective view of portions of the cartridge shown in FIG. 2A, in accordance with certain embodiments of the disclosure.

FIG. 3 schematically shows a heating element, located on a surface of a porous member, for use in the cartridge shown in FIG. 2A in accordance with certain embodiments of the disclosure.

FIG. 4 schematically represents a cross sectional view of a cartridge, for use with the control unit from FIG. 1, in accordance with certain embodiments of the disclosure.

FIG. 5A schematically represents a perspective view of a portion of the cartridge from FIG. 4, for use with the control unit from FIG. 1, in accordance with certain embodiments of the disclosure.

FIG. 5B schematically represents a perspective view of a portion of the cartridge from FIGS. 4 and 5A, for use with the control unit from FIG. 1, in accordance with certain embodiments of the disclosure.

FIGS. 6A and 6B schematically represent a perspective view of a portion of a cartridge with an alternative configuration to FIGS. 4, 5A and 5B, for use with the control unit from FIG. 1, in accordance with certain embodiments of the disclosure.

FIGS. 7A and 7B schematically represent a perspective view of a portion of a cartridge with a further alternative configuration to FIGS. 4, 5A and 5B, for use with the control unit from FIG. 1, in accordance with certain embodiments of the disclosure.

FIG. 8 schematically outlines a suitable composite material (GB-Matrix type Inter-Connector produced by Shin-Etsu Polymer Co., Ltd.) for use in the aerosol provision system of the present disclosure.

FIGS. 9A and 9B schematically represent a perspective view of a portion of a cartridge with a further alternative configuration to FIGS. 7A and 7B, for use with the control unit from FIG. 1, in accordance with certain embodiments of the disclosure.

### DETAILED DESCRIPTION

Aspects and features of certain examples and embodiments are discussed / described herein. Some aspects and features of certain examples and embodiments may be implemented conventionally and these are not discussed / described in detail in the interests of brevity. It will thus be appreciated that aspects and features of apparatus and methods discussed herein which are not described in detail may be implemented in accordance with any conventional techniques for implementing such aspects and features.

The present disclosure relates to non-combustible aerosol provision systems, which may also be referred to as aerosol provision systems, such as e-cigarettes. According to the present disclosure, a “non-combustible” aerosol provision system is one where a constituent aerosolizable material of the aerosol provision system (or component thereof) is not combusted or burned in order to facilitate delivery to a user. Aerosolizable material, which also may be referred to herein as aerosol generating material or aerosol precursor material, is material that is capable of generating aerosol, for example when heated, irradiated or energized in any other way.

Throughout the following description the term “e-cigarette” or “electronic cigarette” may sometimes be used, but it will be appreciated this term may be used interchangeably with aerosol provision system / device and electronic aerosol provision system / device. An electronic cigarette may also known as a vaping device or electronic nicotine delivery system (END), although it is noted that the presence of nicotine in the aerosolizable material is not a requirement.

In some embodiments, the non-combustible aerosol provision system is a hybrid system to generate aerosol using a combination of aerosolizable materials, one or a plurality of which may be heated. In some embodiments, the hybrid system comprises a liquid or gel aerosolizable material and a solid aerosolizable material. The solid aerosolizable material may comprise, for example, tobacco or a non-tobacco product.

Typically, the non-combustible aerosol provision system may comprise a non-combustible aerosol provision device and an article for use with the non-combustible aerosol provision device. However, it is envisaged that articles which themselves comprise a means for powering an aerosol-generating component may themselves form the non-combustible aerosol provision system.

In some embodiments, the article for use with the non-combustible aerosol provision device may comprise an aerosolizable material (or aerosol precursor material), an aerosol generating component (or vaporizer), an aerosol generating area, a mouthpiece, and/or an area for receiving aerosolizable material.

In some embodiments, the aerosol-generating component is a vaporizer or heater capable of interacting with the aerosolizable material so as to release one or more volatiles from the aerosolizable material to form an aerosol. In some embodiments, the aerosol-generating component is capable of generating an aerosol from the aerosolizable material without heating. For example, the aerosol generating component may be capable of generating an aerosol from the aerosolizable material without applying heat thereto, for example via one or more of vibrational, mechanical, pressurization or electrostatic means.

In some embodiments, the substance to be delivered may be an aerosolizable material which may comprise an active constituent, a carrier constituent and optionally one or more other functional constituents.

The active constituent may comprise one or more physiologically and/or olfactory active constituents which are included in the aerosolizable material in order to achieve a physiological and/or olfactory response in the user. The active constituent may for example be selected from nutraceuticals, nootropics, and psychoactives. The active constituent may be naturally occurring or synthetically obtained. The active constituent may comprise for example nicotine, caffeine, taurine, theine, a vitamin such as B6 or B12 or C, melatonin, a cannabinoid, or a constituent, derivative, or combinations thereof. The active constituent may comprise a constituent, derivative or extract of tobacco or of another botanical. In some embodiments, the active constituent is a physiologically active constituent and may be selected from nicotine, nicotine salts (e.g. nicotine ditartrate/nicotine bitartrate), nicotine-free tobacco substitutes, other alkaloids such as caffeine, or mixtures thereof.

In some embodiments, the active constituent is an olfactory active constituent and may be selected from a “flavor” and/or “flavorant” which, where local regulations permit, may be used to create a desired taste, aroma or other somatosensorial sensation in a product for adult consumers. In some instances, such constituents may be referred to as flavors, flavorants, cooling agents, heating agents, and/or sweetening agents. They may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gas, one or more of extracts (e.g., licorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder.

In some embodiments, the flavor comprises menthol, spearmint and/or peppermint. In some embodiments, the flavor comprises flavor components of cucumber, blueberry, citrus fruits and/or redberry. In some embodiments, the flavor comprises eugenol. In some embodiments, the flavor comprises flavor components extracted from tobacco. In some embodiments, the flavor may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucalyptol, WS-3.

The carrier constituent may comprise one or more constituents capable of forming an aerosol. In some embodiments, the carrier constituent may comprise one or more of glycerine, glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

The one or more other functional constituents may comprise one or more of pH regulators, coloring agents, preservatives, binders, fillers, stabilizers, and/or antioxidants.

As noted above, aerosol provision systems (e-cigarettes) often comprise a modular assembly including both a reusable part (control unit) and a replaceable (disposable) cartridge part. Devices conforming to this type of two-part modular configuration may generally be referred to as two-part devices. It is also common for electronic cigarettes to have a generally elongate shape. For the sake of providing a concrete example, certain embodiments of the disclosure described herein comprise this kind of generally elongate two-part device employing disposable cartridges. However, it will be appreciated the underlying principles described herein may equally be adopted for other electronic cigarette configurations, for example modular devices comprising more than two parts, as devices conforming to other overall shapes, for example based on so-called box-mod high performance devices that typically have a more boxy shape.

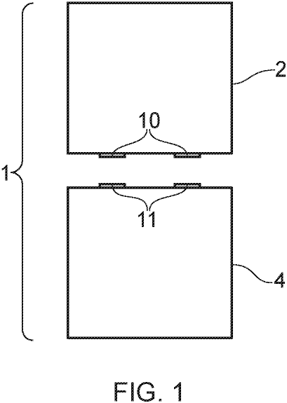

FIG. 1 is a schematic perspective view of an example aerosol provision system / device (e-cigarette) 1 in accordance with certain embodiments of the disclosure. Terms concerning the relative location of various aspects of the electronic cigarette (e.g. terms such as upper, lower, above, below, top, bottom, etc.) are used herein with reference to the orientation of the electronic cigarette as shown in FIG. 1 (unless the context indicates otherwise). However, it will be appreciated this is purely for ease of explanation and is not intended to indicate there is any required orientation for the electronic cigarette in use.

The e-cigarette 1 comprises two main components, namely a cartridge 2 and a control unit 4. The control unit 4 and the cartridge 2 are coupled together when in use.

The cartridge 2 and control unit 4 are coupled by establishing a mechanical and electrical connection between them. The specific manner in which the mechanical and electrical connection is established is not of primary significance to the principles described herein and may be established in accordance with conventional techniques, for example based around a screw thread, bayonet, latched or friction-fit mechanical fixing with appropriately arranged electrical contacts / electrodes for establishing the electrical connection between the two parts as appropriate. For example, in the case of the cartridge 2 shown in FIG. 1, this cartridge 2 comprises a mouthpiece end 6 and an interface end 8. The cartridge 2 is coupled to the control unit 4 by a coupling arrangement (not shown in the Figures) at the interface end 8 of the cartridge 2 such to provide a releasable mechanical engagement between the cartridge and the control unit. An electrical connection is established between the control unit and the cartridge via a pair of electrical contacts/electrodes 10 on the bottom of the cartridge 2 and corresponding contact pins/electrodes 11 in the control unit 4. As noted above, the specific manner in which the electrical connection is established is not significant to the principles described herein. In accordance with a particular embodiment, the control unit 4 may comprise a cartridge receiving section that includes an interface arranged to cooperatively engage with the cartridge 2 so as to releasably couple the cartridge 2 to the control unit 4. In this way, electrical power from the control unit 4 may be delivered to the cartridge via the electrode 10 from the cartridge 2.

It will be appreciated the specific size and shape of the electronic cigarette and the material from which it is made is not of primary significance to the principles described herein and may be different in different implementations. That is to say, the principles described herein may equally be adopted for electronic cigarettes having different sizes, shapes and / or materials.

The control unit 4 may in accordance with certain embodiments of the disclosure be broadly conventional in terms of its functionality and general construction techniques. In some embodiments, the control unit may comprise a plastic outer housing including a receptacle wall that defines a receptacle for receiving the interface end 10 of the cartridge 2.

The control unit 4 further comprises a power supply, such as a battery for providing operating power for the electronic cigarette 1, control circuitry for controlling and monitoring the operation of the electronic cigarette, a user input button, and a charging port.

The battery in some embodiments may be rechargeable and may be of a conventional type, for example of the kind normally used in electronic cigarettes and other applications requiring provision of relatively high currents over relatively short periods. The power supply/battery may be recharged through the charging port, which may, for example, comprise a USB connector.

The input button may be considered an input device for detecting user input, e.g. to trigger aerosol generation, and the specific manner in which the button is implemented is not significant. For example, other forms of mechanical button or touch-sensitive button (e.g. based on capacitive or optical sensing techniques) may be used in other implementations, or there may be no button and the device may rely on a puff detector for triggering aerosol generation.

The control circuitry is suitably configured / programmed to control the operation of the electronic cigarette to provide conventional operating functions in line with the established techniques for controlling electronic cigarettes. The control circuitry (processor circuitry) may be considered to logically comprise various sub-units / circuitry elements associated with different aspects of the electronic cigarette’s operation. For example, depending on the functionality provided in different implementations, the control circuitry may comprises power supply control circuitry for controlling the supply of power from the power supply/battery to the cartridge in response to user input, user programming circuitry for establishing configuration settings (e.g. user-defined power settings) in response to user input, as well as other functional units / circuitry associated functionality in accordance with the principles described herein and conventional operating aspects of electronic cigarettes. It will be appreciated the functionality of the control circuitry can be provided in various different ways, for example using one or more suitably programmed programmable computer(s) and / or one or more suitably configured application-specific integrated circuit(s) / circuitry / chip(s) / chipset(s) configured to provide the desired functionality.

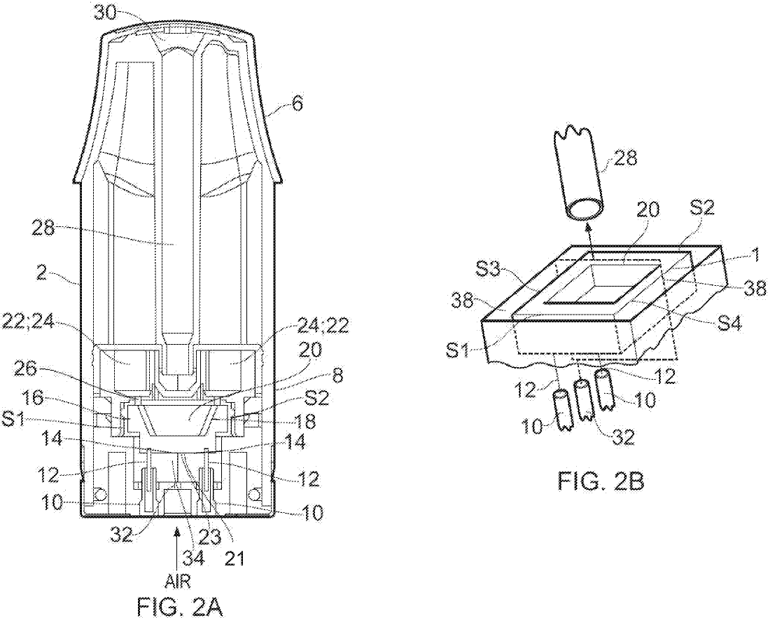

FIG. 2A schematically represents a cross sectional view of a cartridge, for use with the control unit from FIG. 1, in accordance with certain embodiments of the disclosure. In general terms, the cartridge comprises the electrodes 10, wherein each electrode 10 comprises an associated lead 12 which is operable to transfer power between the electrode 10 and a heating element 14. The cartridge 2 may further comprise a porous member 16 for use in holding a fluid to be atomized using the heating element 14. As shown in FIG. 2A, the porous member 16 may comprise a recess 18 defining a basin 20 for holding the fluid. In some embodiments, the porous member 16 may be a ceramic material, and may comprise silicone.

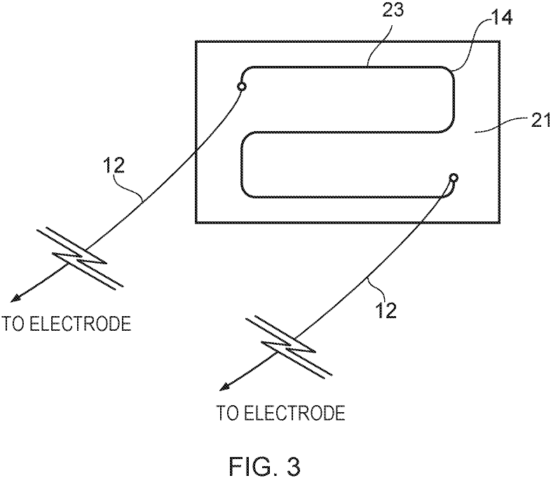

In the embodiment shown in FIG. 2A, the heating element 14 is located between the basin 20 and each electrode 10. In terms of the structure of the heating element 14, in some embodiments the heating element 14 may be located on a surface 21 of the porous member 16. In the case of the embodiments shown in FIGS. 2A and 3, the surface 21 is located on an opposite side of the porous member to that of the basin 20.

To improve the transfer of heat from the heating element to the porous member 16, in some embodiments the heating element 14 may comprise a metal wire or some other conductive material, which may form a tortuous path 23 on the surface 21 of the porous member 16. In that arrangement, a first end of the heating element may be connected to one of the two leads 12, and a second end opposite the first end of the heating element connected to the other of the two leads 12. In terms of the exact shape of the heating element 14, it will be appreciated that the heating element 14 in such embodiments may take any required shape on the surface of the porous member 16 for efficiently vaporizing the aerosolizable material/fluid in the porous member 16. In that respect, and in accordance with some particular embodiments, the heating element/vaporizer 14 may define a spiral pattern; a raster pattern; or a zig-zag pattern on the surface of the porous member 16.

Located towards the mouthpiece end 6 of the cartridge is a chamber 22 acting as a primary reservoir 24 for storing fluid to be aerosolized. The chamber 22 is connected to the basin 20 via at least one opening 26 for topping up the level of fluid in the basin 20, which acts a secondary reservoir.

Extending through the center of the chamber 22 is an outlet channel 28 for receiving aerosol generated from fluid emanating from the porous member 16. The outlet channel 28 extends from the porous member up towards a mouthpiece 30 located at the mouthpiece end 6 of the cartridge, for allowing a user to inhale the aerosol which is generated.

The cartridge comprises an air channel 32 extending through the cartridge for delivering air to the heating element 14. In the embodiment shown in FIG. 2A, the air channel 32 is located between the electrodes 10. Upon connection of the cartridge 2 with the control unit 4, the electronic cigarette 1 would be provided with a further air channel located in the cartridge 2 and/or the control unit 4 which is in fluid communication with the air channel 32, and which is configured to allow ambient air to be passed there through and into air channel 32.

The heating element 14 is located in an aerosol generation region 34 from the cartridge 2, and the outlet channel 28 and the air channel 32 are connected to the aerosol generation region 34.

In normal use, the cartridge 2 is coupled to the control unit 4 and the control unit activated to supply power to the cartridge 2 via the electrodes 10;11. Power then passes through the connection leads 12 to the heating element 14.

The function of the porous member 16 is to act as a capillary wick for drawing fluid from the basin 20 to the heating element 14. Accordingly, fluid which is wicked towards the heating element 14 through the porous member 16 is vaporized by the heat generated from the heating element 14. The generated vapor emanates from the surface 21 where it mixes with the air from the air channel 32 in the aerosol generation region 34 to form an aerosol. Fluid which is vaporized from the porous member 16 is replaced by more fluid drawn from the chamber 22 via the at least one opening 26.

Air enters the air channel 32 as a result of the user inhaling on the mouthpiece 30 of the cartridge 2. This inhalation causes air to be drawn through whichever further air channel aligns with the air channel 32 of the cartridge. The incoming air mixes with aerosol generated from the heating element 14 to form a condensation aerosol at the underside of the porous member 16 in the aerosol generation region 34. The formed aerosol then passes from the underside of the porous member 16, past a gap 38 located on two sides S3;S4 of the porous member as shown in FIG. 2B (the sides S3;S4 being perpendicular to the sides S1;S2 shown in FIG. 2A), and then up through the outlet channel 28 to the mouthpiece 30.

The above therefore describes a cartridge 2 for an aerosol provision system, wherein the cartridge 2 comprises a heating element/vaporizer 14 located in an aerosol generation region 34 from the cartridge 2, and is for heating/vaporizing fluid from a reservoir 20;24 to generate aerosol in the aerosol generation region 34, wherein the cartridge 2 further comprises an air channel 32 extending through the cartridge 2 for delivering air to the heating element/vaporizer 14.

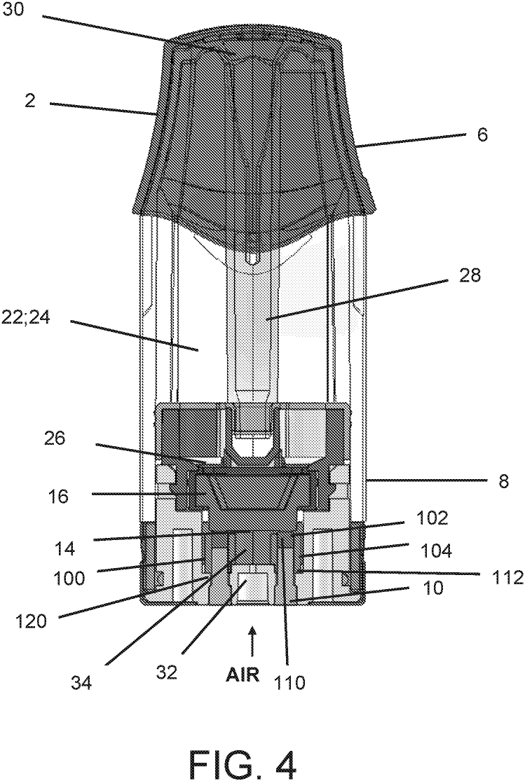

With reference to FIGS. 4-7B, 9A and 9B, there are schematically shown modified cartridges 2 or portions thereof for use with the control unit 4 shown in FIG. 1 to form an aerosol provision system 1 in accordance with certain embodiments of the disclosure. The cartridge 2, or portions thereof, shown in FIGS. 4-7B, 9A and 9B are based on the construction of cartridge 2 shown in FIGS. 1-3, and comprise similar components as set out by the reference numerals that are common to both sets of Figures. For instance, the cartridge 2 comprises the at least one electrode 10, the heating element/vaporizer 14, and the porous member 16.

A principal modification to the cartridge 2 shown in FIGS. 4-7B, 9A and 9B over the cartridge shown in FIG. 2A-3 is the introduction of a sealing member 100 to replace all or part of the connection lead 12. In this respect, the connection leads 12 may become detached from the electrode 10 during use, causing unwanted short-circuits and faulty operation of the cartridge 2. A potential further drawback is that with such connection leads 12, which are shown in FIGS. 2A-2B as embedded in the electrode 10, fluid/vapor (e.g. aerosolizable material/generated aerosol) may ingress in the gap between the connection lead 12 and the electrode 10, which may impact on the efficiency in any electrical power transmitted between the connection lead 12 and the electrode 10, e.g. as a result of corrosion, particularly galvanic corrosion, forming in this gap.

From the foregoing therefore, and as will be described, the disclosure from FIGS. 4-7B, 9A and 9B effectively provides an aerosol provision system 1 comprising a vaporizer 14 for generating a vapor from an aerosolizable material; an electrode 10 for receiving electrical power; and a sealing member 100, electrically connected to the vaporizer 14 and the electrode 10, for transferring electrical power between the electrode 10 and the vaporizer 14, wherein the sealing member 100 is at least partially composed of a heat-resistant and electrically conductive composite material. As will be described, via the introduction of this sealing member 100, this may notionally alleviate the aforementioned disadvantages caused by use of the connection lead(s) 12.

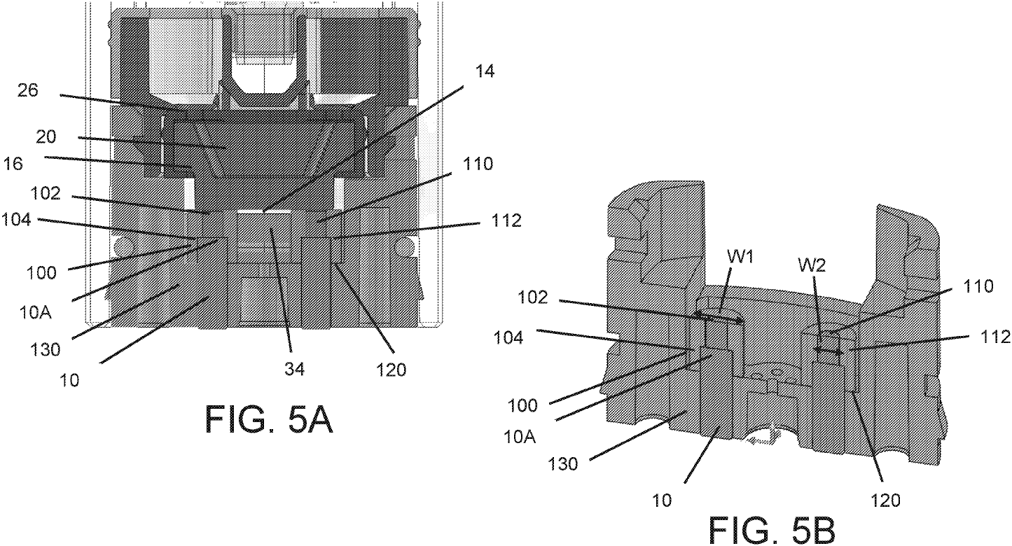

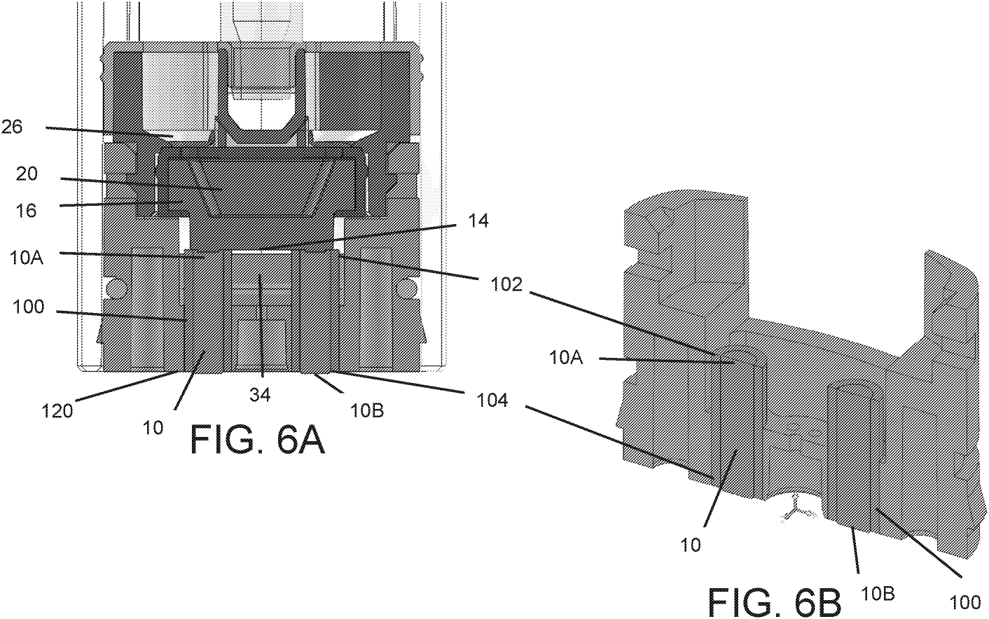

Mindful of the above, and starting with the disclosure from FIG. 4-5B, the sealing member 100 may be provided with a first portion 102 proximal to the vaporizer 14 and a second portion 104 proximal to the electrode. In accordance with such embodiments, such as that shown in FIGS. 4, 5A and 5B, the first portion 102 may effectively be in contact with the vaporizer 14 and the second portion 104 in contact with the electrode 10. The first portion 102 and second portion 104 may respectively also be in contact with the electrode 10/vaporizer 14 and together extend around the first end of the electrode 10. As shown in FIGS. 4, 5A and 5B, the sealing member 100 may therefore be in the form of a cap with the electrode 10 located within a recess of the cap and the cap extending around the first end 10A of the electrode. In this configuration, and in the embodiments of FIGS. 6A, 6B, 7A, 7B, 9A and 9B where the sealing member 100 similarly has a/at least one location for an electrode, the sealing member 100 prevents fluid/vapor (e.g. any aerosolizable material which has inadvertently leaked from porous member 16 into the aerosol generation region 34 and the generated aerosol) from coming into contact with the electrode 10 whilst also ensuring that electrical power is transferred from the electrode 10 to the vaporizer 14 by virtue of the heat-resistant and electrically conductive composite material. The prevention or reduction of fluid/vapor coming into contact with the electrode prevents or reduces the level of corrosion, particularly galvanic corrosion, within the aerosol provision system and thereby reduces the level of metal which may be present in the aerosol inhaled by a user.

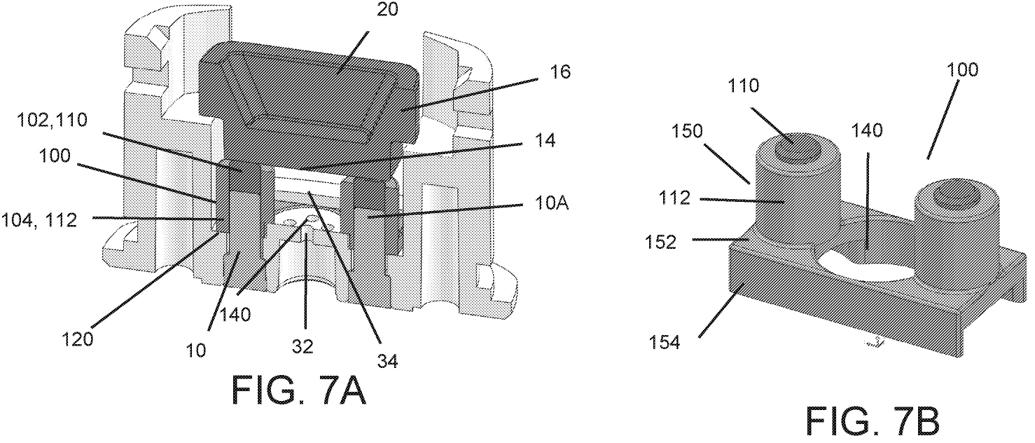

The configuration of the cap in FIGS. 4 and 5A-5B and the embodiment of FIGS. 7A and 7B has a core component 110 and a shell component 112, but the present disclosure is not limited in this respect. In such embodiments where the sealing member is a cap or any other suitable shape, the member could, for instance, be formed of a single material, namely the heat-resistant, electrically conductive composite material defined herein.

Staying with the configuration of the sealing member 100 and the electrode 10, in various embodiments of the present disclosure the sealing member 100 comprises a location for the electrode 10, configured to surround at least the first end of the electrode. This location may be a recess or cover defined in or by the sealing member 100 and allow the sealing member 100 to engage and/or encapsulate electrode 10. The engagement or encapsulation of the electrode 10 by the sealing member 100 not only restricts any unwanted movement/slip of the electrode 10, but it provides a barrier against contact of fluid/vapor with the electrode 10, specifically the corrodible metal(s) of the electrode.

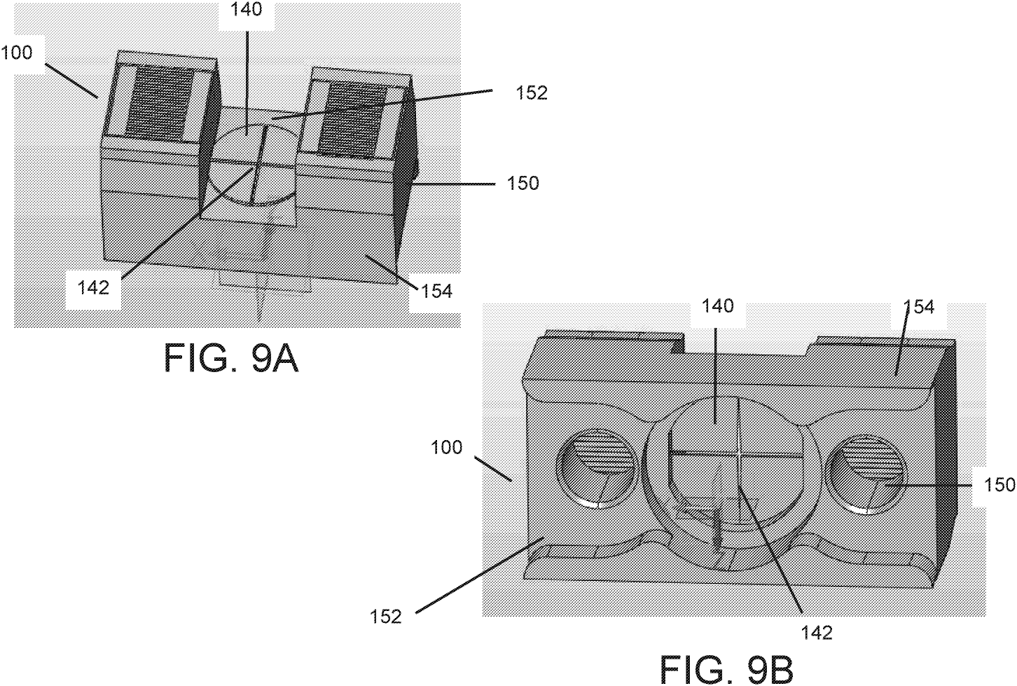

As discussed above, the location for the electrode may be provided by a recess of a cap. In the embodiments of FIGS. 5A and 5B, for instance, the location is provided by a recess in a second portion 104 (shell component 112) of the sealing member 100. In this manner, the first portion 102 (core component 110) of sealing member 100 rests directly on the electrode 10 and the second portion 104 extends around (e.g. concentrically) at least the first end 10A thereof. The second portion 104 further extends around (e.g. concentrically) the first portion 102, thereby resulting in the “core/shell” configuration discussed herein. Where an aerosol provision system includes multiple or a plurality of electrodes 10, there may be a corresponding number of sealing members 100 (as shown in the embodiments of FIGS. 5A-5B and 6A-6B). Alternatively, the sealing member 100 may include multiple or a plurality of locations for the number of electrodes 10 present (as shown in the embodiment of FIGS. 7A-7B and the embodiment of FIGS. 9A-9B). The latter is discussed in more detail below.

In the embodiment shown in FIGS. 6A and 6B, the sealing member 100 includes a first portion 102 proximal to the vaporizer 14 and a second portion 104 proximal to a surface 120 of the aerosol provision system, for example a surface of the cartridge interfacing with the control unit of FIG. 1 (not shown) and adjacent to the second end 10B of electrode 10. In the configuration of FIGS. 6A and 6B, the sealing member 100 thereby extends along the length of the electrode 10 from a first end 10A to a second end 10B and takes the form of a jacket or outer layer for the electrode 10. In that respect, and in accordance with some embodiments, the sealing member 100 may comprise a location for the electrode which is a primary recess in the jacket. In accordance with some particular embodiments, such as that shown in FIGS. 6A-6B, the jacket has a first portion 102 in contact with the vaporizer 14, and a second portion 104 opposite to the first portion 102 which is in contact with a surface 120 of the aerosol provision system, the surface being adjacent to the second end 10B of electrode 10. In this manner, the jacket extends around the circumference and along the length of the electrode 10, and forms a seal or barrier against any fluid/vapor (e.g. aerosolizable material and/or aerosol) present in for example, the aerosol generation region 34.

The present disclosure is not, however, limited to this configuration and the person skilled in the art will appreciate that the embodiment shown in FIGS. 6A and 6B may be modified so that the sealing member jacket 100 extends around (e.g. around the circumference of the electrode 10) and partially along the length of the electrode 10. In such a modification, the sealing member 100 may have a first portion 102 proximal to the vaporizer 14 and a second portion 104 proximal to a surface 120 of the aerosol provision system, the surface being adjacent to the electrode 10 at a location along its length. The second portion 104 may be opposite the first portion 102. The surface 120 with which the second portion 104 engages may, for example, be formed by an element in a base part of the aerosol provision system (e.g. a base part of the cartridge), such as an element for holding the electrode 10. In such embodiments, the electrode 10 may be co-molded into the base part of the aerosol provision system. Regardless of whether the sealing member 100 is along the whole or partial length of electrode 10, it forms a protective coat or wrapper around the exposed surface of the electrode (i.e. the surface vulnerable to corrosion) whilst also facilitating the transfer of electrical power to the vaporizer.

Considering the geometry of the electrode further, in at least some embodiments (such as those shown in FIGS. 4-7B, 9A and 9B), the electrode 10 may extend between a first end 10A and a second end 10B, wherein the first end 10A is located more proximal to the vaporizer 14 than the second end 10B, and wherein the first end 10A in accordance with some particular embodiments thereof may be located opposite the second end 10B (for instance in the case of the electrode being cylindrical). In accordance with such geometry, this may allow for a convenient spacing and positioning of the electrode 10 relative to the vaporizer 14 and the sealing member 100.

Where any surface feature(s) is present in or on the electrode 10, it will be appreciated that any such feature(s) may facilitate the sealing member 100 to engage with the electrode 10. Such surface feature(s) of electrode 10 may, for instance, correspond with feature(s) of the location (e.g. recess) in the sealing member 100 for the electrode. Similarly the sealing member 100 may comprise a keyed surface (not shown) to engage with the electrode 10. Such a surface may prevent the electrode 10 from moving or rotating within the aerosol provision system during assembly thereof, e.g. during assembly of the cartridge or cartomizer. This restriction of electrode movement helps to prevent surface damage to the electrode and hence reduces the susceptibility of the electrode to corrosion. The shape of the keyed surface may appreciably take any required shape to achieve this effect, the keyed surface may for example comprise a flat surface, a castellated surface, or comprise a recess and/or projection for engaging with a corresponding projection and/or recess in the electrode 10.

The present disclosure is also not limited to a cylindrical electrode. In some embodiments, for instance, the cross-sectional area of the electrode 10 may change along its length, e.g. the cross-sectional area of the electrode 10 may decrease in the direction from the second end 10B to the first end 10A or vice versa. Any such decrease in the cross-sectional area may be a progressive decrease in accordance with some embodiments. Additionally and/or alternatively, in accordance with some embodiments, the electrode 10 may be configured to comprise a first section of the electrode 10 comprising a first cross sectional area, and comprise a second section of the electrode 10 comprising a second cross sectional area which is smaller than the first cross sectional area, wherein the second section is located more proximal to the first end 10A and/or the vaporizer 14 than the first section is located to the first end 10A and/or the vaporizer 14. In some particular embodiments thereof, the electrode 10 may further comprise a third section of the electrode 10 comprising a third cross sectional area which is smaller than the second cross sectional area, wherein the third section is located more proximal to the first end 10A and/or the vaporizer 14 than the second section is located to the first end 10A and/or the vaporizer 14. In all such embodiments, it will be understood that the sealing member 100, including the respective portions 102, 104 and components 110, 112 thereof, will have a cross-sectional area which substantially, if not entirely, mirrors that of the electrode 10. As noted above, the sealing member 100 forms a protective coat or barrier around the exposed surface of the electrode 10.

Considering the material of the sealing member 100 in more detail, it is clear from the discussion above that one of the primary functions of the sealing member 100 is to transfer electrical power between the electrode 10 and the vaporizer 14 whilst preventing fluid/vapor from coming into contact with the electrode 10. That being the case, the sealing member 100 is at least partially composed of a heat-resistant and electrically conductive composite material. The terms “heat-resistant” and “electrically conductive” are understood in the art. In the context of the present disclosure, the composite material has the minimum heat-resistance in order to function and maintain its properties at the temperatures typically found in an aerosol provision system such as an e-cigarette. The composite material also has a minimum electrical conductivity so that electrical power is transferred from the electrode to the vaporizer.

In various embodiments, the term “heat-resistant” means that the composite material is capable of resisting temperatures up to about 300° C. When the composite material includes silicone, such as silicone rubber, this material is, for example, known to be resistant to temperatures from -55 to 300° C. while still maintaining its useful properties. Heat-resistance may be measured in accordance with JIS K 6229 by looking at the hardness, elongation at break, tensile strength and/or volume resistivity of the material over a period of time (e.g. 30 days at 5 day intervals) and at different temperatures (e.g. 150, 200 and 250° C.). A material is heat-resistant if the hardness, elongation at break, tensile strength and volume resistivity does not show statistically significant change at the temperature of interest.

In various embodiments, the term “electrically conductive” means that the composite material is able to transport electrical power or charge. Suitable measurement methods are known in the art. As discussed in more detail below, the composite material may be compressible meaning that the material is pressure-sensitive and reduces in volume or size under pressure. In various embodiments, compression of the sealing member lowers the electrical resistance of the composite material and hence increases electrical conductance. In other words, the composite material may have a resistance of X with no compression and a resistance of Y under compression; X/Y may equal the degree to which the sealing member has been compressed (e.g. 10%). The composite material may be a solid or a gel, typically a solid.

As is known in the art, a composite material is a material made from two or more constituent materials with significantly different physical or chemical properties that, when combined, produce a material with characteristics different from the individual components. The individual components remain separate and distinct within the finished structure, thereby differentiating composites from mixtures and solid solutions. Composites are made up of individual materials referred to in the art as “constituent materials”. There are two main categories of constituent materials: matrix materials and reinforcement materials. At least one portion of each type is required. The matrix material surrounds and supports the reinforcement materials by maintaining their relative positions, whilst the reinforcements impart their special mechanical and physical properties to enhance the matrix properties. In various embodiments of the present disclosure, the composite material - referring to both the matrix and reinforcement materials therein -comprises at least one ceramic, polymer, carbon fiber, metal, metal alloy or a combination thereof. In various embodiments, the composite material comprises a ceramic such as silicone, a carbon fiber or a combination thereof. In various embodiments, the composite material comprises a metal, metal alloy or a combination thereof.

The metal or metal alloy may be in any form, for example, in the form of wires, flakes, beads, spheres or the like, and may be a plated material, for example, a plated alloy or a plated metal including gold or silver-plated brass or nickel. The metal or metal alloy is further not limited and can include any known metal or electrically conductive metal alloy in the art. The metal or metal alloy may, for example, include silver, gold, platinum, palladium, nickel, iron, tin, cobalt, cadmium, zinc, chromium, manganese, copper, aluminum, titanium, or salts or combinations thereof. The metal alloy may, for example, be stainless steel, brass, or the like.

In various embodiments of the present disclosure, the composite material is selected from the group consisting of: a ceramic matrix composite, a metal matrix composite, or a combination thereof. Ceramic matrix composites typically consist of ceramic fibers embedded in a ceramic matrix; both the matrix and fibers can consist of any ceramic material, whereby carbon and carbon fibers can be considered a ceramic material. Carbon, silicon carbide, alumina, and mullite fibers are most commonly used for ceramic matrix composites. The use of carbon fibers increases the electrical conductivity of such materials.

Such ceramic matrix composite (CMC) materials can be prepared using methods known in the art, e.g. matrix deposition from a gas phase, matrix formation via pyrolysis of carbon and silicon-containing polymers, matrix forming via chemical reaction, matrix forming via sintering, or matrix forming via electrophoresis. Suitable materials are also commercially available, for example: the EC Series from Shin-Etsu Polymer Co., Ltd. are Electrically Conductive Silicone Rubber Products which have the qualities of silicone rubber plus electrical conductivity from the addition of carbon and other conductive materials. ShinEtsu EC-BL may, for example be used. A composite material such as ShinEtsu EC-BL may be particularly beneficial for the embodiment shown in FIGS. 5A and 5B, either as the material for the core component 110 or the material for both the core 110 and shell 112 components.

A metal matrix composite is a composite material with at least two constituent parts, one being a metal necessarily; the other material may be a different metal or another material, such as a ceramic or organic compound (e.g. a polymer). When at least three materials are present, it is called a hybrid composite. Metal matrix composites (or MMCs) are made by dispersing a reinforcing material into a metal matrix. The reinforcement surface can be coated to prevent a chemical reaction with the matrix. The metal of the metal matrix composite is defined above.

MMCs suitable for the present disclosure can be prepared using methods known in the art or are commercially available. Manufacturing techniques can be divided into three types: solid-state methods, liquid-state methods and vapor deposition. Commercially available materials include, for example: Inter-Connector Materials produced by Shin-Etsu Polymer Co., Ltd. (Shin-Etsu Inter-Connector™) such as GB-Matrix type Inter-Connectors which consist of multiple rows of metal wires (e.g. gold-plated brass wires) embedded in a sheet of insulating silicone rubber. The Shin-Etsu GB-Matrix type Inter-Connector may be particularly useful in the embodiment of FIGS. 6A and 6B or the modification thereof discussed herein where the sealing member is integrally formed with the electrode.

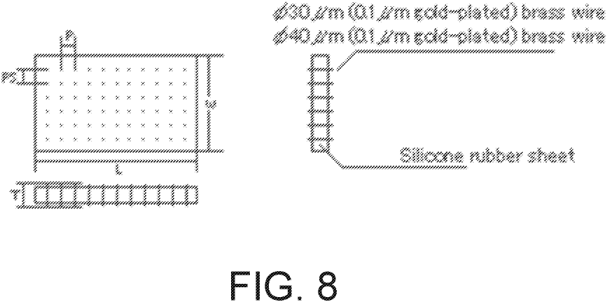

A schematic outline of the Shin-Etsu GB-Matrix type Inter-Connector material is shown in FIG. 8 along with approximate locations for the wires in the rubber sheet. The letters in FIG. 8 refer to P = Pitch or Length direction, PS = Pitch or Width Direction, L = Length, W=Width, and T=Thickness.

Another suitable commercially available material is the MS-type of Inter-Connector produced by Shin-Etsu Polymer Co., Ltd. This type of Inter-Connector consists of alternating conductive and non-conductive layers of silicone rubber. Conductivity is provided by dispersed conductive silver particles in the conductive layers. A material of this type is shown schematically in FIGS. 9A and 9B. In accordance with such material, the sealing member may comprise a layer of the composite material (e.g. a metal matrix composite as defined herein) and a layer of a different material (e.g. an electrically insulating material such as silicone). Notably such a sealing member is not limited to the embodiment shown in FIGS. 9A and 9B, it may, for instance, be used as a cap as shown in FIGS. 5A and 5B or a jacket as shown in FIGS. 6A and 6B.

In various embodiments, the sealing member 100 is composed at least partially of a metal matrix composite, a ceramic matrix composite material or a combination thereof. This material may comprise silicone.

In various embodiments, the composite material may be compressible. By the term “compressible” is meant that the volume of the composite material can change when pressure is applied. The level of compression is not limited and typically depends on the composite material being used in the sealing member. In various embodiments, the compression of the sealing member may reduce the volume of the composite material by about 1% to about 40%. In various embodiments the compression of the sealing member may reduce the volume of the composite material by about 1% to about 25%, for example by about 5% to about 15%; noting that compression of the sealing member may facilitate one of its key functions, namely electrical conductivity, since it can lower the electrical resistance of the composite material. Without wishing to be bound by any one theory, providing compression of the composite material may decrease the space between conductive material (e.g. dispersed conductive silver particles) and thereby realize a stable connection.

The compressibility of the composite material in various embodiments of the present disclosure, allows at least the first portion 102 of sealing member 100 to be held in compression by the vaporizer 14. The first portion 102 may, for instance, be held in compression between the vaporizer 14 (or porous member 16) and the electrode 10. The second portion 104 of sealing member 100 may also be held in compression by the vaporizer 14 or porous member 16 (where present), for example, between the vaporizer 14 and the electrode 10 and/or between the vaporizer 14 and a surface 120 of the aerosol provision system. The location of surface 120 is not limited but in many cases, it is adjacent to the electrode 10. As discussed above with reference to FIGS. 6A and 6B, the surface 120 may be adjacent to the electrode 10 at a location along its length, and be, for example, formed by an element in a base part of the aerosol provision system, such as an element for holding the electrode 10. It can be seen from FIGS. 5A and 5B that the surface 120 configured to engage with second portion 104 of sealing member 100 is opposite to the vaporizer 14 and formed by an element 130 which accommodates the base of electrode 10. In such embodiments, the electrode 10 may be co-molded with the base part of the aerosol provision system.

Such compression can further allow the sealing member 100 to at least partly support the vaporizer 14 and the porous member 16 (where present) during use. In particular, the sealing member 100 may be held in compression between the vaporizer 14 and the electrode 10 and/or between the vaporizer 14 and a surface 120 of the aerosol provision system. The surface 120 is discussed above; such a surface 120 may be located adjacent to the electrode 10 at a position distal from the vaporizer, for example, in a base part which holds the electrode 10 and optionally forms the interface with the control part 4 of FIG. 1 (not shown).

Focusing now on the core/shell configuration of FIGS. 5A and 5B, it can be seen how the sealing member 100 has a core component 110 and a shell component 112. These components form a cap as discussed above, although other forms are within the scope of this disclosure (e.g. a jacket or the like). In various embodiments, the core component 110 and shell component 112 may be composed of different materials, although at least one component is at least partially composed of the heat-resistant, electrically conductive composite material defined herein. The identity of the other component is not, however, limited provided that electrical power is transferred from the electrode 10 to the vaporizer 14 by the sealing member 100.

The core component 110 may, for example, be composed (at least partially) of the heat-resistant, electrically conductive composite material, whilst the shell component 112 is composed of an electrically insulating material (e.g. a silicone material). This arrangement and choice of materials may be used to reduce manufacturing costs and reduce the area of conductive material in contact with the vaporizer. The latter may be beneficial to avoid shortening the heater and reducing the heating effect.

With continued reference to FIGS. 4, 5A and 5B, the core component 110 may be proximal to the electrode 10. The core component 110 may in fact be in contact with the electrode 10, specifically a first end 10A thereof. The core component 110 may further be proximal, and even in contact with the vaporizer 14 thereby providing support as well as electrical contact between the vaporizer 14 and electrode 10. In the embodiments of FIGS. 4, 5A and 5B, the core component 110 is located directly between the vaporizer 14 and electrode 10 but the person skilled in the art will understand that there may be further elements between the core component 110 and/or vaporizer 14. Where the core component 14 is composed of the heat-resistant, electrically conductive composite material, these elements must also be electrically conductive to allow electrical power to flow from the electrode 10 to the vaporizer 14.

The shell component 112 may be proximal to the vaporizer 14 and proximal to the electrode 10. The shell component 112 may in fact be in contact with the vaporizer 14 and in contact with the electrode 10. As discussed above, the shell component 112 may have a recess for locating the electrode 10, specifically the first end 10A of the electrode 10, therein, and extend circumferentially around the first end 10A and core component 110.

As already discussed, the sealing member 100 may be configured to support (at least partially or fully) the vaporizer 14 and/or the porous member 16 (if present). In that way, and in accordance with some embodiments, the core component 110 may be configured to be held in compression between the vaporizer 14 and the electrode 10. Alternatively or additionally, the shell component 112 may be configured to be held in compression between the vaporizer 14 and electrode 10, and/or between the vaporizer 14 and a surface 120 of the aerosol provision system, where the surface may be adjacent to the electrode as discussed hereinabove with reference to FIGS. 4, 5A, 5B, 6A and 6B.

An additional and/or alternative variant of the core-shell configuration is shown by the embodiment of FIGS. 7A and 7B. In this embodiment the sealing member 100 has the same core component 110 as in FIGS. 5A and 5B, but the shell component 112 extends to cover both electrodes in the form of a cover or a saddle and there is a core component 110 for each electrode 10. Put another way, the aerosol provision system includes a single sealing member 100 with a location for each electrode 10 instead of a sealing member 100 for each electrode 10. The saddle form of the sealing member increases its surface area since it includes a bridge between each electrode location, and this can improve the reduction/prevention of galvanic corrosion within the device. The core component 110 and shell component 112 can have the same features as discussed above in respect of FIGS. 5A and 5B; notably the core component 110 can be composed of the composite material defined herein and the shell component 112 can be composed of a different material, for example, an electrically insulating material. The core component 110/shell component 112 may further be held in compression by the vaporizer 14, such as between the vaporizer 14 and electrode 10 and/or between the vaporizer 14 and a surface 120 of the aerosol provision system.

Staying with the embodiment shown in FIGS. 7A and 7B, the sealing member 100 may be described as a cover or cap having a plurality of locations 150 for a plurality of electrodes 10 and a cavity 140 defining an air channel upstream of the vaporizer. The plurality of locations for the plurality of electrodes may be defined as recesses in which the electrodes are received. An alternative configuration for the sealing member 100 having a plurality of locations 150 for the plurality of electrodes 10 and a cavity 140 is shown in FIGS. 9A and 9B.

The sealing member 100 in each of the embodiments of FIGS. 7A, 7B, 9A and 9B is configured to receive the electrodes 10 and accommodate the air channel 32. Given that a primary purpose of the sealing member 100 is to cover the electrodes 10 and prevent fluid/vapor contact with the corrodible metal thereof, it will be appreciated that the shape of the sealing member 100 could take any required form to achieve this functionality where the shape will depend on the configuration of the electrodes 10 and air channel 32 in the aerosol provision system.

In accordance with some particular embodiments, the locations 150 for the electrodes 10 might include a shell component 112 with a substantially cylindrical cross-section (as shown by the embodiment of FIG. 7B) and a core component 110 in contact with each electrode 10, sitting atop the first end 10A thereof. To facilitate electrical contact between the sealing member 100, electrodes 10 and vaporizer 14, each core component 110 may protrude from the surface of the shell component 112. This core component 110 may be held in compression by the vaporizer as discussed above in relation to the embodiment of FIGS. 5A and 5B. To assist the fit and orientation of the sealing member 100 in the aerosol provision system of FIGS. 7A-7B, 9A-9B, the bridge 152 between electrode locations 150 may have one or more side portions 154 for keeping the sealing member 100 in place and in contact with a surface of the aerosol provision system. The side portions 154 may, for instance, ensure that the sealing member 100 has an interference fit with a surface 120 of the aerosol provision system, namely a surface adjacent to the electrode and optionally in a base part of the device. The side portions 154 may further be configured to engage against a portion of the system, such as a wall of the air channel 32.

In accordance with some other embodiments, the locations 150 for the electrodes 10 in the sealing member 100 might include a recess or opening with a substantially cylindrical cross-section (as shown by the embodiment of FIGS. 9A and 9B), and a composite material positioned at one end of the recess/opening, for example across an end of the opening as can be seen clearly from the embodiment of FIG. 9B. The sealing member 100 in such embodiments comprises a layer or a plurality of layers of the composite material and a layer or plurality of layers of a different material, for example an electrically insulating material. This material is discussed generally above. The insulating layers may be positioned substantially in between the layers of composite material and thereby act as a heatsink, reducing the risk of any damage to the composite from overheating or the like.

In a similar manner to the embodiment shown in FIGS. 7A and 7B, the embodiment of FIGS. 9A and 9B includes a bridge 152 between electrode locations 150 with one or more side portions 154 for keeping the sealing member 100 in place and in contact with a surface of the aerosol provision system (or cartridge). The side portions 154 may further be configured to engage against a portion of the system, such as a wall of the air channel 32.

With continued reference to FIGS. 7A-7B and 9A-9B, the sealing member 100 further comprises a cavity 140 between the locations for the electrodes 10. This cavity 140 allows for air flow upstream of the vaporizer 14 and in the air channel upstream of the aerosol generation region 34. With reference to this cavity 140, a valve 142 may be introduced (an embodiment with a valve is shown in FIGS. 9A and 9B). The function of the valve 142 is to allow air to pass into the aerosol generation region 34 upon a user inhalation at the mouthpiece outlet/aerosol outlet 30, but inhibit aerosol generated inside the aerosol generating region 34 from flowing through the air channel back towards the air inlet. The valve 142 may also assist in preventing aerosolizable material from leaking from the base of the aerosol provision system (cartridge). The valve 142 may be any type of one-way valve of a suitable size and operating characteristic for the particular aerosol provision system. In some embodiments the valve 142 may be a reed valve or a duckbill valve.

In accordance with some embodiments, and as shown by the embodiment of FIG. 9A and FIG. 9B, the valve 142 may be integrally formed with the sealing member 100. In this way, as opposed to having the valve 142 formed as a separate component to the sealing member 100, the overall number of separate components in the aerosol provision system can be reduced. As shown in FIGS. 9A and 9B, in some cases the valve 142 may have one or more sections which taper inwardly in a direction extending away from an interface end of the sealing member, and such that it tapers inwardly inside the aerosol generating region. In this way any aerosol condensing on the valve 142 itself may slide off the valve, which better ensures the valve remains fully operational.

Staying with the desire to reduce the overall number of separate components in the aerosol provision system, the sealing member 100 may be co-molded into the base of the aerosol provision system and thereby provide a reliable electrical connection between the vaporizer 14 and electrode 10 whilst preventing liquid or aerosol coming into contact with the electrode material. As an alternative to the embodiment shown in FIGS. 6A-6B, the sealing member may be integrally formed with the electrode (not shown). For example, the electrode 10 may be partially or completely replaced by sealing member 100 such that electrical power is transferred from the power supply in the control unit to the vaporizer by the sealing member 100, the sealing member being composed at least partially and typically entirely of the heat-resistant, electrically conductive composite material as defined herein.

As to the physical dimension of the sealing member 100 and the electrode 10 herein described, it will be entirely appreciated that these physical dimensions may depend on the intended application of these components and/or any aerosol provision system 1 in which the components are located. In accordance with some embodiments where the aerosol provision system 1 is configured to be handheld or portable, in accordance with some very particular embodiments thereof, the sealing member 100 and/or the electrode 10 may comprise any combination of the following physical dimensions (see FIG. 5B):
i) maximum width W1 of the sealing member 100: no more than 2.5 mm and/or between 1.5 mm and 2.5 mm;
ii) maximum width W2 of the core component 110: no more than 2.0 mm and/or between 0.5 and 2.0 mm; and
iii) maximum height of the core component 110: no more than 2.0 mm and/or between 0.5 and 2.0 mm.

With respect to the sealing member 100 described herein and as illustrated in the embodiments from FIG. 4-7B, it is envisaged (as noted previously) that this sealing member 100 may be used with some of the other previously described features of the aerosol provision system 1 described with reference to FIGS. 1-3, such as but not limited the porous member 16, the vaporizer 14, and any of the other features from the cartridge 2 or control unit 4 shown in FIGS. 1-3 which collectively form the aerosol provision systems 1 described herein.

Accordingly, there has been described an aerosol provision system comprising: a vaporizer for generating a vapor from an aerosolizable material; an electrode for receiving electrical power; and a sealing member, electrically connected to the vaporizer and the electrode, for transferring electrical power between the electrode and the vaporizer, wherein the sealing member is at least partially composed of a heat-resistant and electrically conductive composite material.

There has also been described a cartridge for an aerosol provision system comprising the cartridge and a control unit, wherein the cartridge comprises: a vaporizer for generating a vapor from an aerosolizable material; an electrode for receiving electrical power from the control unit; and a sealing member, electrically connected to the vaporizer and the electrode, for transferring electrical power between the electrode and the vaporizer, wherein the sealing member is at least partially composed of a heat-resistant and electrically conductive composite material.

For the sake of completeness however, it is to be noted that the sealing member 100 described herein need not be expressly used in an aerosol provision system 1 which comprises a cartridge 2 and the control unit 4. Accordingly, the sealing member 100 may be notionally used in any aerosol provision system 1 which is configured to generate a vapor from an aerosolizable material.

Also in respect of the sealing member 100 described herein, it will be appreciated that there may be provided one or more sealing members 100, as required, depending on how many electrodes 10 there are. Accordingly, although the description has been principally described with reference to the operation of a single sealing member 100, it will be appreciated (as noted in FIGS. 4, 5A, 5B, 6A and 6B) that more than one sealing member 100 may in practice be employed, as required, such as there being one sealing member for each provided electrode 10. In that respect as well, and purely for the avoidance of any doubt, where more than one sealing member 100 is provided, the plurality of sealing members 100 may all electrically connect to a single vaporizer 14 and/or electrically connect to a separate vaporizer 14 for each electrode 10, depending on the particular application of the sealing member 100. In that respect, and with reference to the embodiment shown in FIGS. 4, 5A, 5B, 6A and 6B, there may in accordance with some particular embodiments be provided an aerosol provision system 1 comprising the vaporizer 14 for generating a vapor from an aerosolizable material; a plurality of electrodes 10 for receiving electrical power; and a plurality of sealing members 100, wherein each sealing member 100 is electrically connected to the vaporizer 14 and a respective one of the electrodes 10, for transferring electrical power between the respective one of the electrodes 10 and the vaporizer 14, and wherein each sealing member 100 is at least partially composed of a heat-resistant and electrically conductive composite material.

In order to address various issues and advance the art, this disclosure shows by way of illustration various embodiments in which the claimed invention(s) may be practiced. The advantages and features of the disclosure are of a representative sample of embodiments only, and are not exhaustive and/or exclusive. They are presented only to assist in understanding and to teach the claimed invention(s). It is to be understood that advantages, embodiments, examples, functions, features, structures, and/or other aspects of the disclosure are not to be considered limitations on the disclosure as defined by the claims or limitations on equivalents to the claims, and that other embodiments may be utilized and modifications may be made without departing from the scope of the claims. Various embodiments may suitably comprise, consist of, or consist essentially of, various combinations of the disclosed elements, components, features, parts, steps, means, etc. other than those specifically described herein, and it will thus be appreciated that features of the dependent claims may be combined with features of the independent claims in combinations other than those explicitly set out in the claims. The disclosure may include other inventions not presently claimed, but which may be claimed in future.

For instance, although the present disclosure has been described with reference to a “liquid” or “fluid” in the cartridge / aerosol provision system, it will be appreciated that this liquid or fluid may be replaced with any aerosolizable material. Equally, where an aerosolizable material is used, it will be appreciated that in some embodiments this aerosolizable material may comprise a liquid or fluid.

Furthermore, whilst the present disclosure has been described with reference to a heater/heating element being present in the cartridge / aerosol provision system, it will be appreciated that in accordance with some embodiments this heating element may be replaced with a vaporizer or some other aerosol-generating component. Equally, such an aerosol-generating component in accordance with some embodiments may in particular comprise a heater or heating element.

1-40. (canceled)

41. An aerosol provision system comprising:
a vaporizer for generating a vapor from an aerosolizable material;
an electrode for receiving electrical power; and
a sealing member that extends around and partially along a length of the electrode,
wherein the electrode extends between a first end and a second end, and the first end is located more proximal to the vaporizer than the second end.

42. The aerosol provision system according to claim 41, wherein the sealing member is at least partially composed of a heat-resistant and electrically conductive composite material.

43. The aerosol provision system according to claim 41, wherein the sealing member is electrically connected to the vaporizer and the electrode, for transferring electrical power between the electrode and the vaporizer.

44. The aerosol provision system according to claim 41, wherein the sealing member has a first portion in proximity to the vaporizer and a second portion in proximity to the electrode or a surface of the aerosol provision system.

45. The aerosol provision system according to claim 41, wherein the sealing member has a maximum width of no more than 2.5 mm.

46. The aerosol provision system according to claim 41, wherein the sealing member has a maximum width of between 1.5 mm and 2.5 mm.

47. The aerosol provision system according to claim 41, wherein the aerosol provision system comprises a plurality of electrodes for receiving electrical power and a plurality of sealing members, wherein each of the plurality of sealing members is electrically connected to the vaporizer and a respective one of the plurality of electrodes for transferring electrical power between the respective one of the plurality of electrodes and the vaporizer.

48. The aerosol provision system according to claim 42, wherein the composite material of the sealing member comprises at least one of ceramic, polymer, carbon fiber, metal, metal alloy, or a combination thereof.

49. The aerosol provision system according to claim 42, wherein the composite material is selected from the group consisting of: a ceramic matrix composite, a metal matrix composite, and a combination thereof.

50. The aerosol provision system according to claim 49, wherein the composite material is a metal matrix composite.

51. The aerosol provision system according to claim 42, wherein the composite material comprises silicone.

52. The aerosol provision system according to claim 41, wherein the sealing member is in the form of a jacket that extends at least partially around the electrode.

53. The aerosol provision system according to claim 52, wherein a location for the electrode is a primary recess in the jacket.

54. The aerosol provision system according to claim 52, wherein the jacket extends around and along at least a length of the electrode.

55. The aerosol provision system according to claim 41, further comprising a porous member for use in holding aerosolizable material to be vaporized using the vaporizer.

56. The aerosol provision system according to claim 41, wherein the vaporizer comprises a heating element.

57. The aerosol provision system according to claim 41, further comprising a reservoir for aerosolizable material, wherein the vaporizer is configured to receive the aerosolizable material from the reservoir.

58. The aerosol provision system according to claim 41, further comprising a cartridge and a control unit, wherein the electrode, the vaporizer, and the sealing member are located in the cartridge, wherein the control unit comprises a cartridge receiving section that includes an interface arranged to cooperatively engage with the cartridge so as to releasably couple the cartridge to the control unit, and wherein the control unit further comprises a power supply for delivering electrical power to the electrode for powering the vaporizer.

59. A cartridge for an aerosol provision system comprising the cartridge and a control unit, the cartridge comprising:
a vaporizer for generating a vapor from an aerosolizable material;
an electrode for receiving electrical power from the control unit; and
a sealing member that extends around and partially along a length of the electrode,
wherein the electrode extends between a first end and a second end, and wherein the first end is located more proximal to the vaporizer than the second end.
